(19) United States
(12) Patent Application Publication
(10) Pub. No.: US 2023/0277104 A1
(43) Pub. Date: Sep. 7, 2023

(54) Blood Collection Device for Small Volume Sample Acquisition Through Peripheral Intravenous Catheters

(71) Applicant: Becton, Dickinson and Company, Franklin Lakes, NJ (US)

(72) Inventors: Erica E. Neumann, Sandy, UT (US); John M. Lackey, West Valley City, UT (US); Benjamin P. Hopwood, Salt Lake City, UT (US); Jonathan Karl Burkholz, Salt Lake City, UT (US); Weston F. Harding, Lehi, UT (US); Shaun Lauer, Portland, OR (US)

(21) Appl. No.: 18/117,748

(22) Filed: Mar. 6, 2023

Related U.S. Application Data
(60) Provisional application No. 63/317,325, filed on Mar. 7, 2022.

(51) Int. Cl. A61B 5/15; A61M 5/31; A61M 25/00; A61M 5/315; A61B 5/153

(52) U.S. Cl. CPC A61B 5/150992; A61M 5/3129; A61M 25/0017; A61M 5/31513; A61B 5/153

(57) ABSTRACT

Provided herein is a blood collection device having a tube having a proximal end, a distal end, and a sidewall therebetween defining an interior, the interior configured to receive a blood sample through one or more openings at the distal end of the tube when the distal end of the tube is positioned in a blood vessel and an obturator slidably disposed relative to the tube and received within the interior of the tube.

### CROSS-REFERENCE TO RELATED APPLICATION

The present application claims priority to U.S. Provisional Application Ser. No. 63/317,325, entitled “Blood Collection Device for Small Volume Sample Acquisition Through Peripheral Intravenous Catheters” filed Mar. 7, 2022, the entire disclosure of which is hereby incorporated by reference in its entirety.

### BACKGROUND OF THE INVENTION

### Field of the Invention

The present disclosure relates generally to devices for use with intravenous (IV) catheter assemblies and, more specifically, to devices for collecting small volumes of blood through peripheral intravenous catheters.

### Description of Related Art

Recent developments in the peripheral intravenous catheter (PIVC) market have led to the emergence of technologies designed to facilitate blood draw from an in-dwelling PIVC. These devices have focused on the ability to reliably collect a high-quality sample and reduce hemolysis. The main method by which these devices work is by inserting a guide wire, probe, or tube through the lumen of the catheter. This creates a fluid path through any thrombus or fibrin that may be occluding the tip of the catheter. A syringe or evacuated container can then be used to collect blood samples without needing to subject the patient to additional needle sticks.

Point-of-care (PoC) testing involves portable testing systems that can quickly deliver results using small volume blood samples, such as for glucose testing. Samples are typically collected via finger pricks or through existing vascular access. However, the majority of vascular access devices are not optimized for collection of small samples. Accordingly, there is a need to provide small-volume blood sample collection options from a PIVC in order to meet growing demands for PoC testing.

### SUMMARY OF THE INVENTION

Provided herein is a blood collection device having a tube having a proximal end, a distal end, and a sidewall therebetween defining an interior, the interior configured to receive a blood sample through one or more openings at the distal end of the tube when the distal end of the tube is positioned in a blood vessel and an obturator slidably disposed relative to the tube and received within the interior of the tube.

In certain configurations, the obturator is slidable relative to the tube between a retracted position, in which the obturator blocks the one or more openings at the distal end of the tube, and an extended position in which the one or more openings at the distal end of the tube are not blocked.

In certain configurations, the obturator is slidable relative to the tube between an extended position, in which the one or more openings at the distal end of the tube are not blocked, and an extended position in which the obturator blocks the one or more openings at the distal end of the tube.

In certain configurations, the obturator comprises a support wire. In other configurations, the blood collection device includes a vent plug arranged within the tube interior, configured to allow air to pass therethrough.

In certain configurations, the obturator includes a tip configured to prevent sample contamination within the interior of the tube. Optionally, the tube includes an opening at the proximal end, and wherein the obturator is configured to expel blood from the interior of the tube through the opening at the proximal end of the tube. In certain configurations, the tube includes a coil arranged at the distal end thereof.

Also provided herein is a blood collection device having an outer tube having a proximal end, a distal end, and a sidewall therebetween defining an interior configured to receive a blood sample when the distal end of the tube is positioned in a blood vessel, and an inner tube, received within the interior of the outer tube, and slidably and/or rotatably disposed relative to the outer tube, the inner tube including a proximal end, a distal end, and a sidewall therebetween, wherein the inner tube comprises one or more openings at the distal end thereof.

In certain configurations, the inner tube is slidable relative to the outer tube between a retracted position, in which the outer tube blocks the one or more openings at the distal end of the inner tube, and an extended position in which the one or more openings at the distal end of the inner tube are not blocked.

In certain configurations, the outer tube includes one or more openings at the distal end thereof, and wherein the inner tube is rotatable relative to the outer tube between a first position, in which the one or more openings at the distal end of the outer tube are not aligned with the one or more openings at the distal end of the inner tube, and a second position in which the one or more openings at the distal end of the outer tube are aligned with the one or more opening at the distal end of the inner tube, thereby allowing blood to flow into the interior of the outer tube.

In certain configurations, the blood collection device further includes a vent plug arranged within the tube interior, configured to allow air to pass therethrough. Optionally, the inner tube includes a tip configured to prevent sample contamination within the interior of the tube. The outer tube and/or the inner tube may include a coil arranged at the distal end thereof.

Also provided herein is a blood collection device having a syringe barrel having a proximal end, a distal end, and a sidewall therebetween defining an interior configured to receive a blood sample, a collection tube slidably received within the syringe barrel interior, the collection tube including a proximal end, a distal end having one or more openings therein, and a sidewall therebetween defining a collection tube interior in fluid communication with the interior of the syringe barrel and configured to receive blood therein when the distal end of the collection tube is positioned in a blood vessel, a tube plunger including a proximal end, a distal end, and a sidewall therebetween, the tube plunger being slidably received within the interior of the syringe barrel and coupled with the collection tube, and a sample plunger slidably received within the interior of the syringe barrel and configured to draw blood into the collection tube interior through the distal end of the collection tube.

In certain configurations, the proximal end of the syringe is configured to reversibly couple to an indwelling catheter. The proximal end of the syringe barrel may include a luer connection.

In certain configurations, the tube plunger is coupled to the proximal end of the collection tube. The collection tube and tube plunger may be configured such that displacement of the tube plunger in a distal direction displaces the collection tube distally a corresponding distance.

In certain configurations, the sample plunger may be slidably received within the tube plunger interior. The sample plunger may be configured such that displacement of the sample plunger in a proximal direction causes blood to be drawn through the collection tube into the syringe barrel interior.

In certain configurations, the collection tube includes a plurality of openings at the distal end thereof. The blood collection device may further include a septum at the distal end of the syringe barrel.

In certain configurations, the tube plunger includes one or more air vents at the distal end thereof. The collection tube may include a coil arranged at the distal end thereof.

Also provided herein is a blood collection system having a vascular access device including a vascular access device housing including a proximal end, a distal end, and a sidewall therebetween defining a vascular access device interior, and a collection tube slidably displaced within the vascular access device interior between a retracted position and an extended position, the collection tube including a proximal end, a distal end, and a sidewall therebetween defining a collection tube interior configured to receive blood therein when the distal end is positioned within a blood vessel, and a blood collection device including a housing having a proximal end, a distal end configured to reversibly couple to the vascular access device, and a sidewall therebetween defining a blood collection device interior, the blood collection device interior configured to be placed in fluid communication with the collection tube interior.

In certain configurations, the distal end of the blood collection device is reversibly coupled to the proximal end of the collection tube, such that when the distal end of the collection tube is positioned within a blood vessel, blood flows through the collection tube and into the blood collection device interior.

In certain configurations, the blood collection device includes a septum at the distal end thereof.

In certain configurations, the blood collection device includes a vent at the proximal end thereof, the vent configured to allow air to pass therethrough.

In certain configurations, the vent is arranged in a venting cap that is removably coupled to the proximal end of the blood collection device housing.

In certain configurations, the blood collection device housing is compressible.

In certain configurations, the blood collection device further includes one or more blood stabilization chemicals in the interior thereof.

In certain configurations, the one or more blood stabilization chemicals include one or more of heparin, sodium citrate, and ethylenediaminetetraacetic acid.

In certain configurations, the one or more blood stabilization chemicals are provided in or on a porous substrate arranged at or near the distal end of the housing, such that blood entering the housing through the distal end passes through the porous substrate and the one or more blood stabilization chemicals are released into the blood.

In certain configurations, the porous substrate is an open-cell foam.

In certain configurations, the collection tube includes a coil arranged at the distal end thereof.

In certain configurations, the distal end of the tube has an outer diameter that is the same as the outer diameter of the coil.

In certain configurations, the distal end of the tube comprises a taper.

### BRIEF DESCRIPTION OF THE DRAWINGS

FIG. 1 is a perspective view of a catheter assembly;

FIG. 2A-2B are cross-sectional views of a blood collection device according to non-limiting embodiments as described herein;

FIGS. 3A-3B are cross-sectional views of a blood collection device according to non-limiting embodiments as described herein;

FIGS. 4A-4C are cross-sectional views (4A, 4B) and side views (4C) of a blood collection device according to non-limiting embodiments as described herein;

FIGS. 5A-5E are cross-sectional views of a blood collection device according to non-limiting embodiments as described herein;

FIG. 6 is a cross-sectional view of a blood collection device according to non-limiting embodiments as described herein;

FIG. 7 is a cross-sectional view of a blood collection device according to non-limiting embodiments as described herein;

FIGS. 8A-8B are perspective (8A) and side (8B) views of a blood collection system and device according to non-limiting embodiments as described herein;

FIGS. 9A-9E are cross-sectional (9A and 9E) and side (9B-9D) views of a blood collection device according to non-limiting embodiments as described herein;

FIGS. 10A-10B are side views of a blood collection device according to non-limiting embodiments as described herein; and

FIG. 11 is a side view of a blood collection system according to non-limiting embodiments as described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

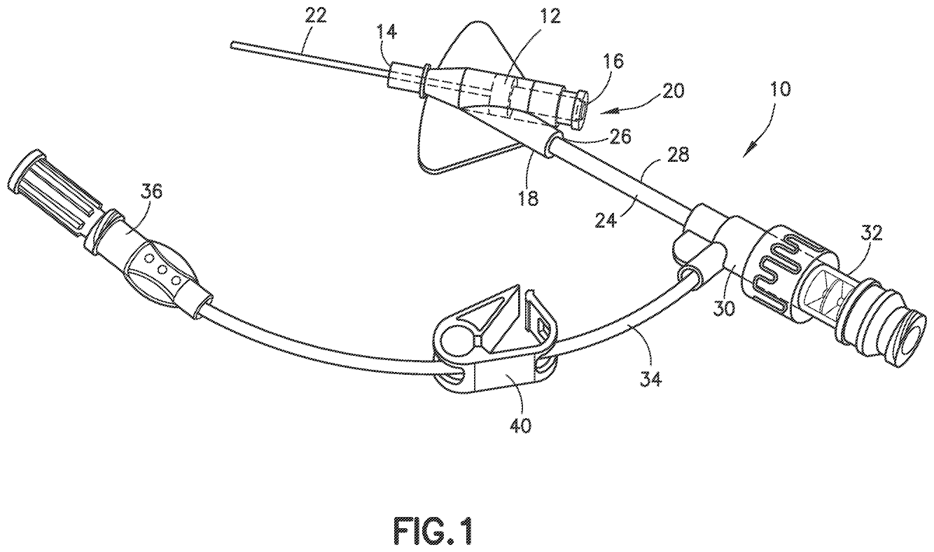

Referring now to FIG. 1, shown is a non-limiting embodiment of a catheter assembly 10, which may include a catheter adapter 12, which may include a distal end 14 and a proximal end 16. In some embodiments, the catheter adapter 12 may include a side port 18 disposed between the distal end 14 and the proximal end 16. In some embodiments, the first catheter adapter 12 may include a first lumen 20 extending through the distal end 14 and the proximal end 16.

In some non-limiting embodiments or aspects, the first catheter assembly 10 may include a first catheter 22 extending from the distal end 14. In some embodiments, the first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the first catheter assembly 10 may include a first fluid conduit 24 extending from the side port 18. First fluid conduit 24 may be formed of any suitable material known to those of skill in the art, and may have a distal end 26 and a proximal end 28, and first fluid conduit 24 may be coupled, at distal end 26 thereof, to side port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at a 25, 60, or 75 degree angle relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set including a second fluid conduit 34. In non-limiting embodiments, connector 30 includes a side port to which an extension set (e.g., second fluid conduit 34) may be connected. Extension sets are known to those of skill in the art and are commercially available from, for example, Becton, Dickinson and Company under the tradenames MAXPLUS, MAXZERO, NEUTRACLEAR, Q-SYTE, and SMARTSITE. In some non-limiting embodiments or aspects, second fluid conduit 34 may include a luer connection 36 at an end thereof. In some non-limiting embodiments or aspects, the extension set may include a clamp 40, to allow for occlusion of second fluid conduit 34. Clamp 40 and second fluid conduit 34 may be formed of any suitable materials known to those of skill in the art. In non-limiting embodiments, second lumen 31 has an inner diameter that is substantially equivalent to an inner diameter of first fluid conduit 24 and/or second fluid conduit 34.

Catheter assembly 10 may include a needleless access connector 32 and/or a second fluid conduit 34. Needleless access connectors are known to those of skill in the art and are commercially available from, for example, Becton, Dickinson and Company under the tradenames SMARTSITE and Q-SYTE.

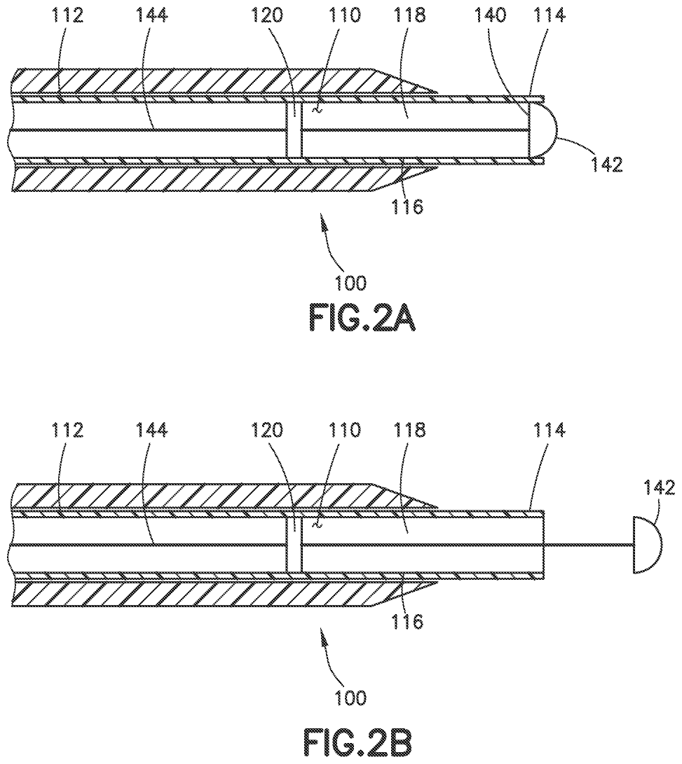

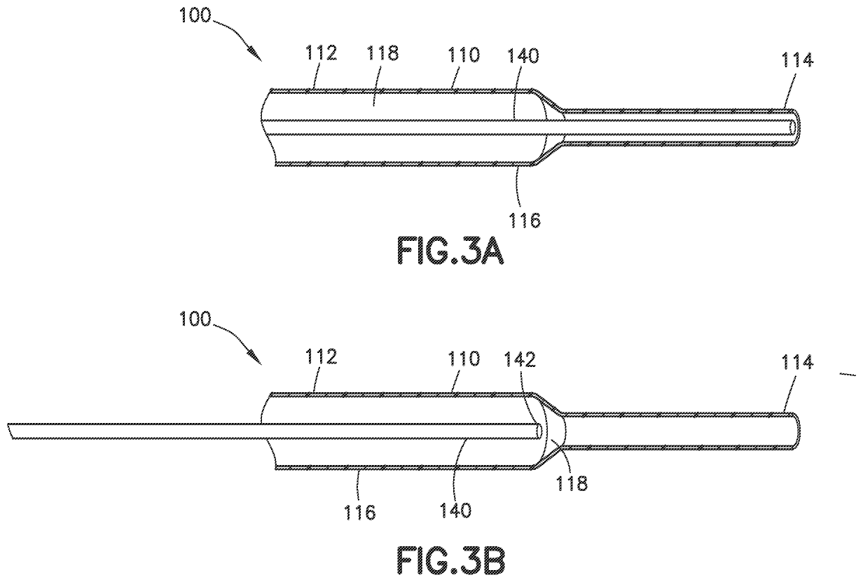

Blood collection devices and systems as described herein, and as exemplified in FIGS. 2A-11, may be couplable, for example reversibly couplable, to one or more components of a catheter assembly 10 as shown in FIG. 1, for example to catheter adapter 12, needless access connector 32, and/or luer connection 36. With reference to FIGS. 2A-3B, shown is a non-limiting embodiment of a blood collection device 100 that may be utilized with an indwelling catheter, for example as shown in FIG. 1. The device as shown in FIGS. 2A-2B may include a collection tube 110 having a proximal end 112, a distal end 114, and a sidewall 116 therebetween defining an interior 118. Interior 118 may be configured to receive a blood sample therein. Collection tube 110 may be formed of any suitable material, for example any suitable flexible polymeric material that can pass through one or more curves in a catheter assembly 10 such as that shown in FIG. 1. In non-limiting embodiments, collection tube 110 may be formed of a polyimide. Collection tube 110 may have a constant outer diameter (OD), or may include a taper of any suitable length/angle.

Blood collection device 100 further includes an obturator 140. Obturator 140 may include a proximal end, a distal end, a tip 142 at the distal end, and a support wire 144 to assist in sliding obturator 140 relative to collection tube 110. Tip 142 of obturator 140 may be configured to aid in traversing blockages in a blood vessel into which blood collection device 100 is positioned, such as clots, thrombi, or the like. Tip 142 may also be configured, for example by the inclusion of one or more sealing members, to prevent contamination, for example to prevent contamination of a sample as blood collection device 100 is positioned within a blood vessel and a sample is obtained, as described below. Once positioned within a blood vessel, obturator 140 may be extended (FIG. 2B), or retracted (FIG. 3B), to place interior 118 of collection tube 110 in fluid communication with one or more openings at or near the distal end 114 of collection tube 110, thereby allowing blood to flow into interior 118.

In non-limiting embodiments, collection tube 110 may include a vent plug 120, configured to allow air to be displaced out of interior 118, which may aid in causing blood to flow into interior 118, and/or pulling blood into interior 118, for example through application of a vacuum due to displacement of air out of interior 118. In non-limiting embodiments, vent plug 120 is liquid impermeable, such that blood cannot flow proximally out of collection tube 110. In this way, obturator 140 can be withdrawn, or extended, to provide a seal at distal end 114 of collection tube 110, allowing for removal of blood collection device 100 from the blood vessel, with a sample held in interior 118.

In non-limiting embodiments, blood collection device 100 is configured to expel the blood sample from the distal end 114 thereof. For example, a medical professional utilizing device 100 to withdraw a small-volume sample from an indwelling catheter, such as a PIVC, can then use obturator 140 as a plunger, similar to that of a standard syringe, to expel blood from the distal end 114 of collection tube 110. In this way, a liquid impermeable vent plug 120 may act as a stopper as typically included in standard syringes.

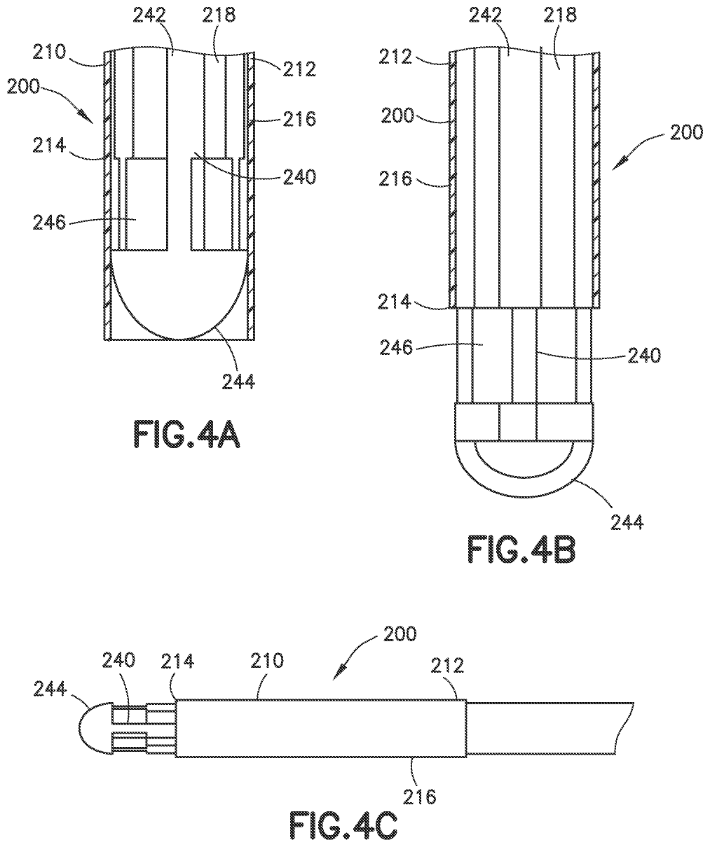

Turning to FIGS. 4A-4C, shown is a non-limiting embodiment of a blood collection device 200. The illustrated non-limiting embodiment may include an outer (e.g., collection) tube 210 having a proximal end 212, a distal end 214, and a sidewall 216 therebetween defining an interior 218. Interior 218 may be configured to receive a blood sample therein. Device 200 may also include an inner tube 240 having a proximal end 242, distal end 244, and one or more openings 246 at or near distal end 244. As shown in FIGS. 4A-4C, distal end of inner tube 240 may include a tip that may function to traverse obstructions, clots, or thrombi, and may prevent contamination of interior 218 of outer tube 210, for example preventing contamination of a sample received within interior 218. Outer tube 210 and inner tube 240 may be formed of any suitable material known to those of skill in the art, for example a flexible polymer to allow device 200 to pass through one or more curves in a catheter assembly such as that shown in FIG. 1. In non-limiting embodiments, outer tube 210 and/or inner tube 240 may be formed of a polyimide. In non-limiting embodiments, outer tube and/or inner tube 240 include a vent plug (not shown), optionally, liquid impermeable, to allow for air to be displaced out of interior 218, thus permitting blood to flow into interior 218.

In non-limiting embodiments, inner tube 240 and outer tube 210 may be slidable and/or rotatable relative to one another. In non-limiting embodiments, inner tube 240 is slidable relative to outer tube 210, such that when distal end 214 of outer tube is positioned in a blood vessel, inner tube 240 may be extended distally. In this way, the one or more openings 246 in inner tube 240 place interior 218 of outer tube 210 in fluid communication with the blood vessel, allowing blood to enter the interior 218.

In non-limiting embodiments, inner tube 240 may be rotatable relative to outer tube 210, and outer tube 210 may include one or more openings at or near distal end 214 thereof. Inner tube 240 may be rotatable between a first position, where one or more openings do not align with one or more openings in outer tube 210, thus blood is blocked from flowing into interior 218, to a second position, where one or more of openings 246 in inner tube 240 are aligned with one or more openings in outer tube 210, thereby placing interior 218 in fluid communication with the blood vessel, allowing blood to enter the interior 218.

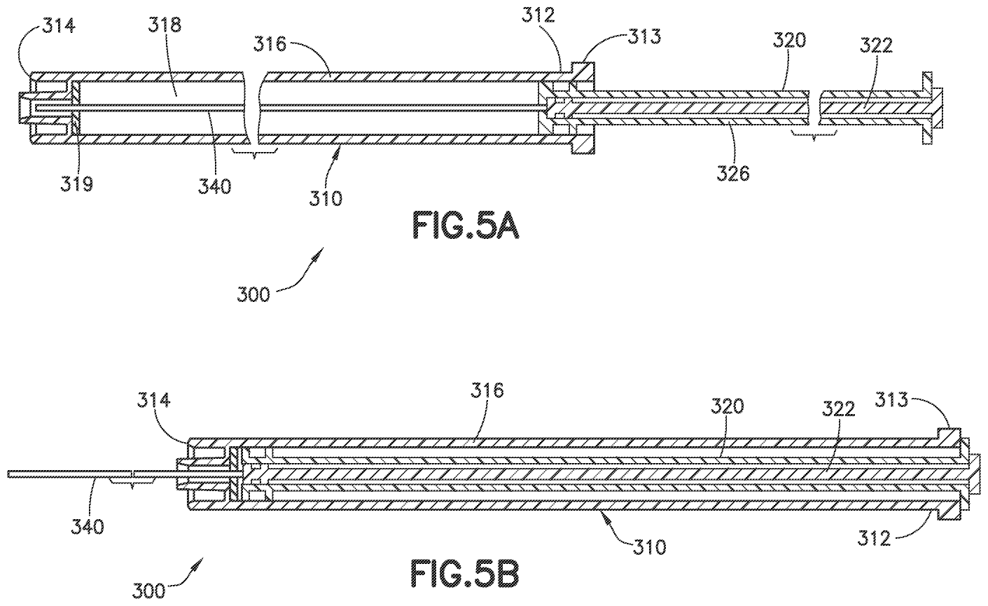

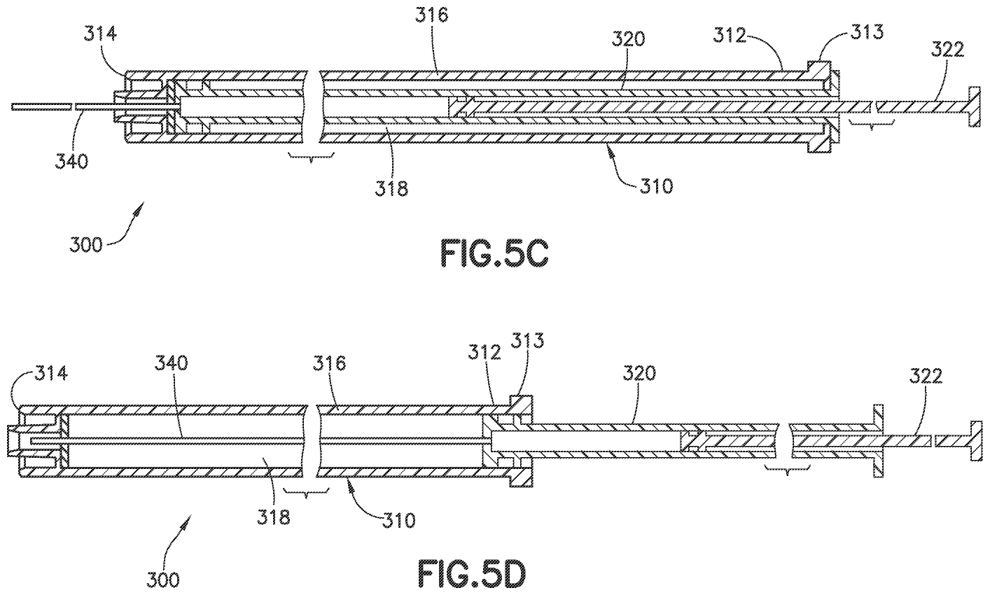

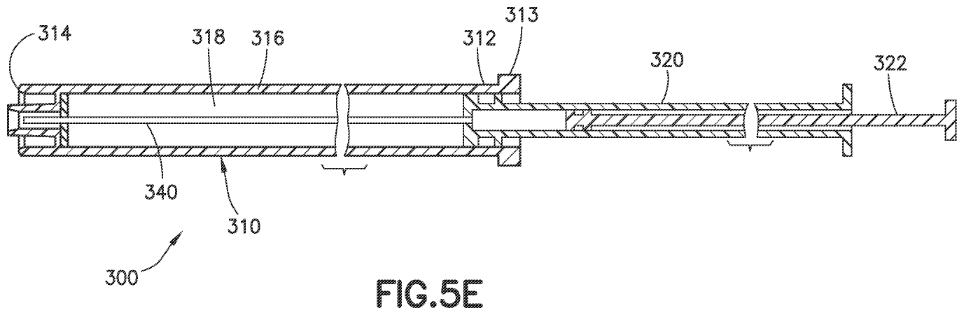

Turning to FIGS. 5A-7, shown is a non-limiting embodiment of a blood collection device 300. In the illustrated embodiment, device 300 is similar to a syringe, and has a syringe barrel 310 with a proximal end 312 optionally having one or more finger flanges 313, distal end 314, and sidewall 316 therebetween defining an interior 318 configured to receive a blood sample. Syringe barrel 310 may include a septum 319 positioned in interior 318, optionally at or near distal end 314. Septum 319 may be a pierceable septum, and may be a fluid-impermeable structure. Syringe barrel 310 may be formed of any useful material, such as glass or plastic, and may include, at distal end 314, one or more features for reversible coupling of barrel 310 to one or more components of a catheter assembly, for example such as that shown in FIG. 1. For example, distal end 314 of syringe barrel 310 may include a luer, a clip, elements to aid in friction fit, and/or other type of connection feature known in the medical arts. Device 300 may further include a collection tube 340 having a proximal end 341, distal end 342, and a sidewall 343 therebetween defining a collection tube interior 344. Collection tube 340 may be formed of any suitable material known to those of skill in the art, for example a flexible polymer to allow collection tube 340 to pass through one or more curves in a catheter assembly such as that shown in FIG. 1.

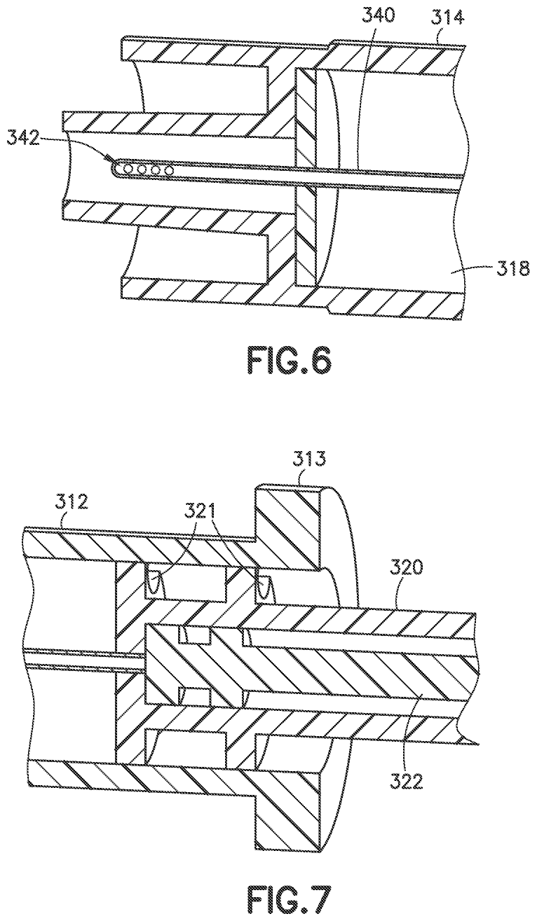

Collection tube 340 may be slidable relative to syringe barrel 310 between a retracted position, in which distal end 342 is within syringe barrel 310 or the distal end 314 of the syringe barrel 310, and an extended positon, in which distal end 342 of collection tube 340 extends beyond syringe barrel 310 or distal end 314 of the syringe barrel 310, for example into a blood vessel. Collection tube may include one or more openings 345 at or near distal end 342 thereof, as shown in FIG. 6, to allow for blood to enter collection tube interior 344; however, those of skill will appreciate that one or more openings 345 may be arranged at one or more positions along sidewall 343.

Device 300 further includes a tube plunger 320, received within syringe barrel interior 318. Tube plunger 320 may be coupled to collection tube 340 and slidable relative to syringe barrel 310, the tube plunger 320 having a proximal end 322, distal end 324, and sidewall 325 therebetween defining an interior 326. Tube plunger 320 may have one or more vents 321 arranged at or near distal end 324 thereof (FIG. 7), to allow air to pass from syringe barrel interior 318 to ambient environment during tube plunger 320 displacement. Tube plunger 320 may be configured to displace collection tube 340 and may, in some embodiments, be coupled to proximal end 341 of collection tube 340. Device 300 may also include a sample plunger 328. Sample plunger 328 may be received within interior 326 of tube plunger 320. Tube plunger 320 and/or sample plunger 328 may include, independently, one or more finger flanges and/or thumb pads to allow a user to manipulate the device 300 as needed to obtain a blood sample, for example as described below.

Device 300 may be utilized to collect small volume blood samples. When distal end 314 of syringe barrel 310 is connected to a catheter, such as one or more components of catheter assembly 10 shown in FIG. 1, both tube plunger 320 and sample plunger 328 may initially be in the fully retracted positon shown in FIG. 5A. Collection tube 340 is then deployed by depressing sample plunger 328, which, by virtue of its placement within interior 326 of tube plunger 320, causes tube plunger, and collection tube 340 coupled thereto, to be displaced distally, placing distal end 342 of collection tube 340 into a blood vessel. Once in position, sample plunger 328 may be withdrawn, as shown in FIG. 5C, causing blood to be pulled into collection tube interior 344 through one or more openings 345 at or near distal end 342 of collection tube 340. Tube plunger 320 may then be withdrawn, as shown in FIGS. 5D-5E, withdrawing collection tube 340, optionally into syringe barrel interior 318, allowing device 300 to be decoupled from the catheter assembly.

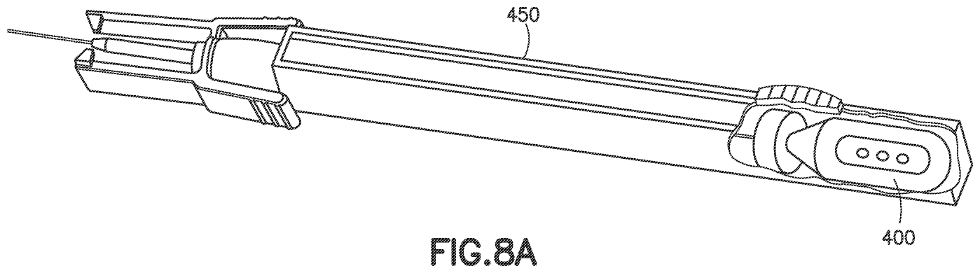

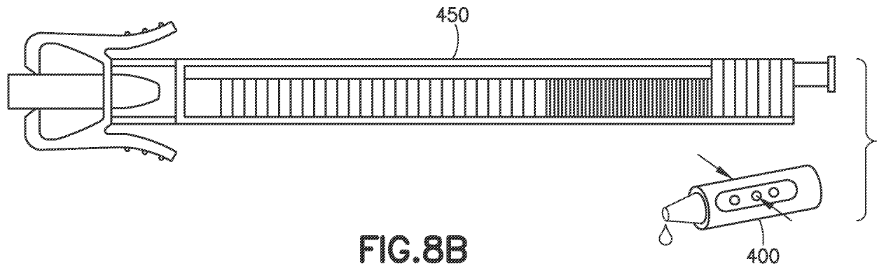

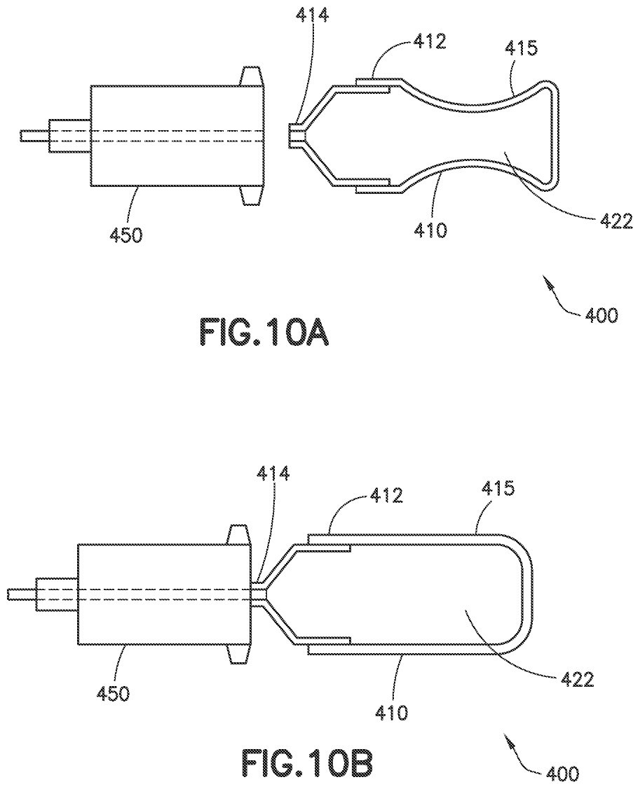

With reference to FIGS. 8-11, shown is a non-limiting embodiment of a blood collection system including a vascular access device 450 and a blood collection device 400. Vascular access device 450 may be the same or similar to that described in U.S. Pat. No. 10,300,247, incorporated herein by reference in its entirety. Blood collection device 400 includes a housing 410 having a proximal end 412, distal end 414, and sidewall 415 therebetween defining an interior 422. Housing interior 422 may be of any suitable shape to promote mixing, for example with one or more chemicals provided therein as described below. Housing 410 may be formed of a resilient material, such that sidewall 415 is compressible, for example by application of a compressive force by a user's fingers, but returns to an expanded state after releasing a compressive force applied thereto. Sidewall 415 may include one or more transparent sections, to allow visual confirmation that a blood sample has been received within interior 422. Distal end 414 may include one or more connection features to allow device 400 to be reversibly coupled to vascular access device 450, such as clips, luer connections, elements to aid in a friction fit, and the like. Blood collection device 400 may include a septum 416 at distal end 414 thereof, and a vent 426 at proximal end 412 thereof. Vent 426 may be configured to let air pass therethrough when housing sidewall 415 is compressed and/or when a blood sample is introduced into interior 422. In non-limiting embodiments, vent 426 is provided on a removably coupled vent cap 424, and vent cap 424 can optionally be removed to allow for the collected blood sample to be dispensed through an opening at proximal end 412 by compressing housing sidewall 415. In non-limiting embodiments, compressing housing sidewall 415 causes the collected blood sample to be dispensed through an opening at distal end 414 of housing 410. Turning to FIGS. 10A-10B, shown in an embodiment of the device 400 in use. FIG. 10A shows an embodiment of a pre-use positon, in which device 400 is not connected to vascular access device 450. A user compresses sidewall 415, expelling air from interior 422. As shown in FIG. 10B, device 400 may then be connected via any suitable connection at distal end 414 thereof, to proximal end of vascular access device 450. A user may then release sidewall 415, as shown in FIG. 10B, creating a draw and pulling blood into housing interior 422. Device 400 may then be disconnected and used, for example by compressing sidewall 415 once again, to transfer the sample to a PoC device

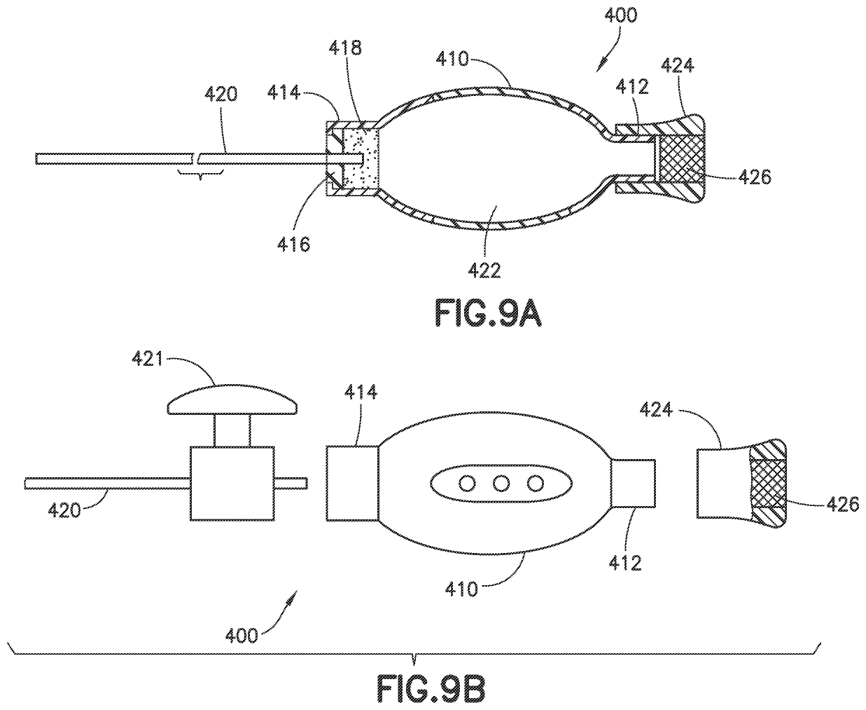

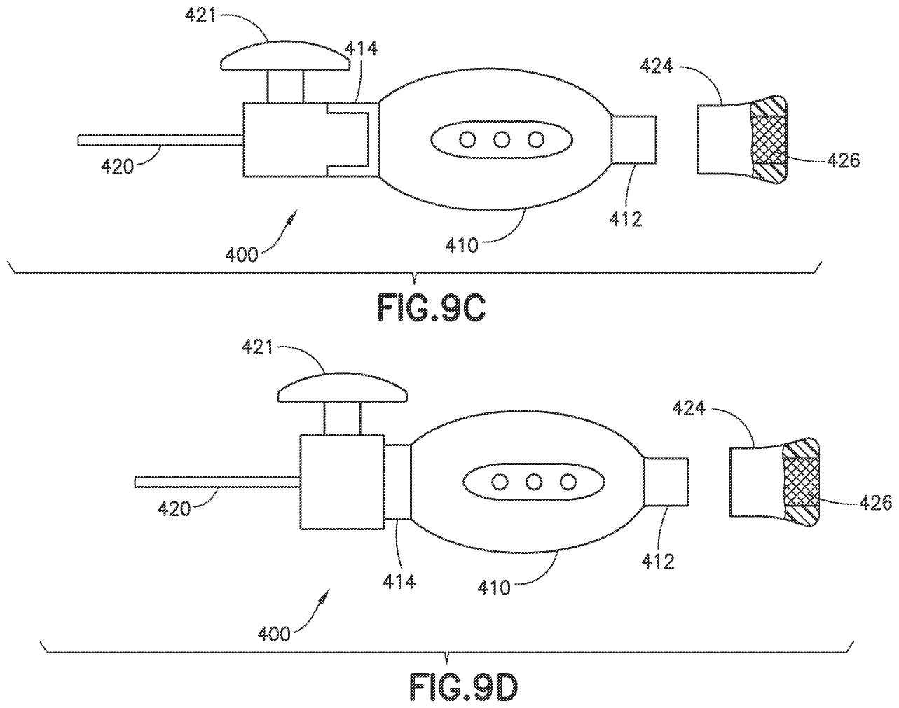

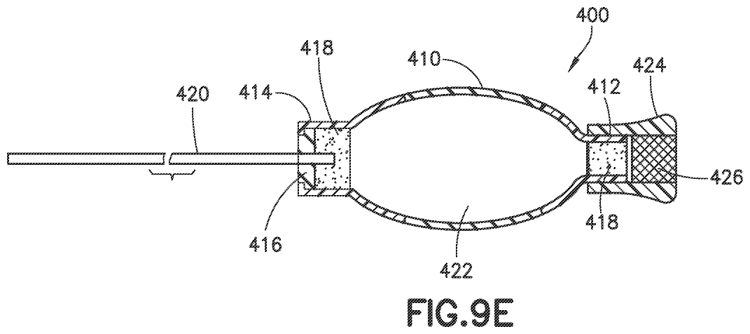

A blood stabilization device may be reversibly coupled to a vascular access device 450, such that a collection tube 420 carried by vascular access device 450 and is slidable relative thereto can be advanced into a blood vessel, for example by a user advancing tab 421 distally. As shown in FIG. 9A, collection tube 420 may be positioned so as to pierce septum 416, and is thus in fluid communication with housing interior 422. Blood received into an interior of collection tube 420 may then pass into housing interior 422 by passive or active venting, for example through venting of air through vent 426. In other embodiments (shown in FIGS. 9C and 9D), tab 421 may be coupled to device housing 410. Tab 421 may be couplable to housing 410 in any suitable manner, for example through a friction fit, a clip, and/or other mechanisms known to those of skill in the art. In non-limiting embodiments, one or more side arms of tab 421 interact with proximal end 414 of housing 410 as shown in FIG. 9C and/or tab 421 interacts with housing 410 via a circumferential fit (by sliding over or into distal end 414 of housing 410), for example as shown in FIG. 9D.

In non-limiting embodiments, device 400 includes one or more blood stabilization chemicals. Blood stabilization chemicals may be coated onto an interior surface of sidewall 415. In non-limiting embodiments, device 400 includes a porous substrate 418, such as an open-cell foam, arranged at or near distal end 414 of housing 410, into and/or onto which one or more blood stabilization chemicals are adhered and/or embedded, such that as a blood sample enters housing interior 422, it flows through the substrate, and the one or more chemicals are then mixed with the blood sample. In non-limiting embodiments, the one or more blood stabilization chemicals are dispersed within the cells of the open cell foam to promote the effectiveness of the flow-through mixing and blood stabilization chemical uptake. In non-limiting embodiments, the open cell foam may be a soft deformable open cell foam that is inert to blood, for example, a melamine foam, such as BASOTECT foam commercially available from BASF (Ludwigshafen, Germany), or may be formed of a formaldehyde-melamine-sodium bisulfite copolymer. The open cell foam may also be a flexible, hydrophilic open cell foam that is substantially resistant to heat and organic solvents. In one embodiment, the foam may include a sponge material. The one or more blood stabilization chemicals may be introduced into the open cell foam by soaking the foam in a liquid solution of the one or more blood stabilization chemicals and water, and subsequently evaporating the water, forming a dry additive powder finely distributed throughout the internal structure of the foam.

In non-limiting embodiments, for example as shown in FIG. 9E, more than one porous substrate 418 may be included in housing interior 422. While FIG. 9E shows a second porous substrate 418 arranged near proximal end of housing 410, those of skill will appreciate that additional porous substrates 418 may be positioned ate any suitable location within housing interior 422. Exemplary arrangements of such porous substrates may be found in U.S. Patent Application Publication No. 2019/0265134, the content of which is incorporated by reference herein in its entirety.

In non-limiting embodiments, one or more beads, coated and/or impregnated with one or more blood stabilization chemicals, can be included in housing interior 422, and can be mixed with a blood sample received therein. Suitable stabilization chemicals are known to those of skill in the art, and can include one or more of heparin, ethylenediaminetetraacetic acid (EDTA), and/or a citrate, such as sodium citrate.

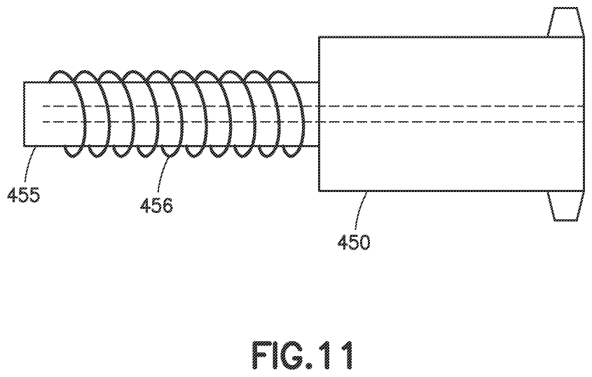

With reference to FIG. 11, in non-limiting embodiments of blood collection systems and devices disclosed herein, the blood collection device, or vascular access device 450 as exemplified in FIG. 11, includes a collection tube 455, such as those described previously. While the illustrated embodiment of FIG. 11 shows a vascular access device 450, those of skill will appreciate that the features provided at the distal end of collection tube 455 may be applied to collection tubes included with blood collection devices as described herein. In non-limiting embodiments, collection tube 455 includes at a distal end thereof a coil 456. Coil 456 may be formed of any suitable material, for example metals and alloys thereof such as nitinol, polymers, or the like, so long as the material is of sufficient rigidity to aid collection tube 455 in passing through an occlusion, thrombus, clot, or like obstruction in a blood vessel into which collection tube 455 is deployed. In non-limiting embodiments, the material is also sufficiently flexible so that unwanted damage to blood vessel walls is avoided or minimized. In non-limiting embodiments, coil 456 is connected to collection tube 455. In non-limiting embodiments, collection tube 455 has the same or similar OD as the coil 456. In non-limiting embodiments, collection tube 455 tapers in a distal direction, from a larger OD than coil 456 to the same or a similar OD as coil 456.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A blood collection device comprising:
a tube having a proximal end, a distal end, and a sidewall therebetween defining an interior, the interior configured to receive a blood sample through one or more openings at the distal end of the tube when the distal end of the tube is positioned in a blood vessel; and
an obturator slidably disposed relative to the tube and received within the interior of the tube.

2. The blood collection device of claim 1, wherein the obturator is slidable relative to the tube between a retracted position, in which the obturator blocks the one or more openings at the distal end of the tube, and an extended position in which the one or more openings at the distal end of the tube are not blocked.

3. The blood collection device of claim 1, wherein the obturator is slidable relative to the tube between an extended position, in which the one or more openings at the distal end of the tube are not blocked, and an extended position in which the obturator blocks the one or more openings at the distal end of the tube.

4. The blood collection device of claim 1, wherein the obturator comprises a support wire.

5. The blood collection device of claim 1, further comprising a vent plug arranged within the tube interior, configured to allow air to pass therethrough.

6. The blood collection device of claim 1, wherein the obturator comprises a tip configured to prevent sample contamination within the interior of the tube.

7. The blood collection device of claim 1, wherein the tube comprises an opening at the proximal end, and wherein the obturator is configured to expel blood from the interior of the tube through the opening at the proximal end of the tube.

8. The blood collection device of claim 1, wherein the tube comprises a coil arranged at the distal end thereof.

9. A blood collection device comprising:
an outer tube having a proximal end, a distal end, and a sidewall therebetween defining an interior configured to receive a blood sample when the distal end of the tube is positioned in a blood vessel; and
an inner tube, received within the interior of the outer tube, and slidably and/or rotatably disposed relative to the outer tube, the inner tube comprising a proximal end, a distal end, and a sidewall therebetween, wherein the inner tube comprises one or more openings at the distal end thereof.

10. The blood collection device of claim 9, wherein the inner tube is slidable relative to the outer tube between a retracted position, in which the outer tube blocks the one or more openings at the distal end of the inner tube, and an extended position in which the one or more openings at the distal end of the inner tube are not blocked.

11. The blood collection device of claim 9, wherein the outer tube comprises one or more openings at the distal end thereof, and wherein the inner tube is rotatable relative to the outer tube between a first position, in which the one or more openings at the distal end of the outer tube are not aligned with the one or more openings at the distal end of the inner tube, and a second position in which the one or more openings at the distal end of the outer tube are aligned with the one or more opening at the distal end of the inner tube, thereby allowing blood to flow into the interior of the outer tube.

12. The blood collection device of claim 9, further comprising a vent plug arranged within the tube interior, configured to allow air to pass therethrough.

13. The blood collection device of claim 9, wherein the inner tube comprises a tip configured to prevent sample contamination within the interior of the tube.

14. The blood collection device of claim 9, wherein the outer tube and/or the inner tube comprises a coil arranged at the distal end thereof.

15. A blood collection device comprising:
a syringe barrel having a proximal end, a distal end, and a sidewall therebetween defining an interior configured to receive a blood sample;
a collection tube slidably received within the syringe barrel interior, the collection tube comprising a proximal end, a distal end having one or more openings therein, and a sidewall therebetween defining a collection tube interior in fluid communication with the interior of the syringe barrel and configured to receive blood therein when the distal end of the collection tube is positioned in a blood vessel;
a tube plunger comprising a proximal end, a distal end, and a sidewall therebetween, the tube plunger being slidably received within the interior of the syringe barrel and coupled with the collection tube; and
a sample plunger slidably received within the interior of the syringe barrel and configured to draw blood into the collection tube interior through the distal end of the collection tube.

16. The blood collection device of claim 15, wherein the proximal end of the syringe is configured to reversibly couple to an indwelling catheter.

17. The blood collection device of claim 15, wherein the proximal end of the syringe barrel comprises a luer connection.

18. The blood collection device of claim 15, wherein the tube plunger is coupled to the proximal end of the collection tube.

19. The blood collection tube of claim 15, wherein the collection tube and tube plunger are configured such that displacement of the tube plunger in a distal direction displaces the collection tube distally a corresponding distance.

20. The blood collection tube of claim 15, wherein the sample plunger is slidably received within the tube plunger interior.

21. The blood collection device of claim 15, wherein the sample plunger is configured such that displacement of the sample plunger in a proximal direction causes blood to be drawn through the collection tube into the syringe barrel interior.

22. The blood collection device of claim 15, wherein the collection tube comprises a plurality of openings at the distal end thereof.

23. The blood collection device of claim 15, further comprising a septum at the distal end of the syringe barrel.

24. The blood collection device of claim 15, wherein the tube plunger comprises one or more air vents at the distal end thereof.

25. The blood collection device of claim 15, wherein the collection tube comprises a coil arranged at the distal end thereof.
